(19) United States
(12) Patent Application Publication
(10) Pub. No.: US 2020/0376249 A1
(43) Pub. Date: Dec. 3, 2020

(54) FLUSHING CATHETER

(71) Applicant: Penumbra, Inc., Alameda, CA (US)

(72) Inventor: Kathleen Lockhart, San Francisco, CA (US)

(73) Assignee: Penumbra, Inc., Alameda, CA (US)

(21) Appl. No.: 16/424,969

(22) Filed: May 29, 2019

(51) Int. Cl. A61M 39/00; A61M 25/00

(52) U.S. Cl. CPC A61M 39/00; A61M 25/0097; A61M 2039/0018; A61M 2025/0006; A61M 25/007

(57) ABSTRACT

The present invention relates to a flushing catheter device, flushing catheter components, and methods for making the same. A flushing catheter includes one or more flush segments comprised of one or more flush ports along a length of catheter body. A preferred method for making a flushing catheter incorporates a retrofit including one or more flush segments. Flushing catheters may be nested within each other's lumens to form a multi-catheter system. Flush segments may enable radial fluid communication between the many lumens and annular lumens of a multi-catheter system, e.g. intraluminal fluid communication. A multi-catheter system including flushing catheters may be entirely purged of air with a single step of fluid injection. A flushing catheter may include a tapered distal end to further enhance the ease of procedure with a multi-catheter system.

### BACKGROUND OF THE INVENTION

The present invention relates to catheterization systems and methods for accessing anatomical spaces in a body, and, more particularly, to the simultaneous flushing of multi-catheter systems.

Many medical procedures utilize catheters. Catheters are typically elongated tubular structures that provide a working channel for accessing a patient's anatomical spaces. Although catheters may be the best and safest treatment option for many diseases, they are not risk free. The working channel of a catheter permits easy access to not only medical devices but also to ambient air. Catheters pose a major risk of introducing an air embolism. For instance, a 15 French (5 mm diameter) catheter sheath open to ambient air, in some scenarios, may allow 300 cc of air to enter the vascular system in only half a second. Although small volumes of air in the venous system may be asymptomatic, only 200 to 300 cc of air in the arterial system may be fatal.

Air embolisms are avoided, at least in part, by priming the catheter(s) with a fluid flush that expels air from any internal cavities of the catheter(s). In one scenario, a patient with an ischemic stroke is admitted to an emergency room. The physician must determine the location of the blood clot(s) and then select appropriately sized catheters to reach it. Each individual catheter must then be removed from its packaging and individually flushed with saline fluid to purge the catheters of air. This is a time-consuming process that takes up valuable time during many life-threatening and time sensitive procedures. In the case of ischemic stroke, the clot is cutting off blood flow to a portion of the brain. Although brain tissue may recover from small time periods of ischemia, an untreated occlusion eventually leads to the death of brain tissue. Thus, every second counts during an ischemic stroke procedure and time spent individually flushing catheters may be time that a patient's brain is suffering irreparable injury.

It is therefore an object of the present invention to improve flushing efficiency for multi-catheter systems, which can accelerate preparation during time sensitive and life-threatening procedures.

### SUMMARY OF THE INVENTION

### 1. Field of the Invention

The present invention is embodied by a catheter or catheter component that includes a flush segment(s) comprised of one or more flush ports. The flush segment(s) is generally positioned along a length of the catheter body. Preferably, the flush segment enables radial fluid communication between a lumen of an inner catheter and an annular lumen of an adjacent or outer catheter, e.g. intraluminal fluid communication.

In one example, the present invention is embodied by a flushing catheter that is integrated with one or more flush segments. (FIG. 1A). In other examples, the present invention is embodied by a retrofit. The retrofit is a catheter subcomponent with one or more flush segments (see FIGS. 4A, 5A, & 6A). The retrofit may be attached to either a catheter body, a catheter hub, or both. Alternatively, the retrofit may be attached to a catheter body on both sides. Once attached, a flushing catheter is formed and the flush segment(s) of the retrofit enables intraluminal fluid communication.

In one use case, an inner flushing catheter is placed inside an outer catheter to form a multi-catheter system. (FIG. 1C). Fluid introduced to the inner catheter through the proximal end has at least two exit paths, either axially out the distal end of the inner flushing catheter or radially through the flush segment and then axially out the distal end of the outer catheter. In this way, a single flush flushes both the inner and outer catheter. Additionally, any number of lumens in a multi-catheter system may be simultaneously flushed so long as all of the inner catheters feature a flush segment according to the present invention.

The novel flush segment(s) of the present invention improves the efficiency of catheterization procedures by reducing the number of preparation steps. A flush segment enables a multi-catheter system to be flushed of air with a single act of flushing through a single injection port. Typically, multi-catheter systems require several steps of flushing, either every catheter is flushed individually and then nested together or every catheter includes an independent injection port and each must be individually flushed. The present invention eliminates the need for individual flushing. The novel flush ports of the present invention enable a single step of flushing to flush two or more catheters simultaneously.

A flushing catheter may be comprised of a catheter body with a length that extends through a proximal region, a central region, and a distal region of the flushing catheter. The catheter body at least partially encloses a lumen that extends between a proximal end and a distal end of the flushing catheter, wherein the lumen has a single injection port. The flushing catheter includes a flush segment having a length and one or more flush ports, wherein the flush segment is located along the length of the catheter body. The flush ports may be embodied by a variety of geometries and configurations. The flush ports may be at least partially restrictive over some types of fluid flow. A flushing catheter may include fastening mechanisms for interlocking to smaller and larger catheters and catheter components.

A flushing catheter subcomponent may be comprised of a fluid channel having a lumen and a length that extend between a first end and a second end. The first end and the second end are configured for attachment to either a catheter hub or a catheter body. The flushing catheter subcomponent includes a flush segment having a length and one or more flush ports, wherein the flush segment is located along the length of the fluid channel. The flushing catheter subcomponent's lumen enables fluid communication at a distal terminus and the flush segment enables fluid communication to, at least, an external space adjacent to and along the length of the flush segment.

A flushing catheter may include a tapered distal end and tip shape that improves navigation in tortuous vasculature. A flushing catheter may include thick walls in at least some regions of the catheter body.

### 2. Listing of the Background Art

Background Art includes U.S. Pat. Nos. 5,207,648; 5,425,723; 5,800,408; and US2004/0097880.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a perspective view of a flushing catheter attached to a fluid injection device.

FIG. 1B illustrates steps to form a multi-catheter system.

FIG. 1C illustrates a partially transparent perspective view of a two-catheter system attached to a fluid injection device.

FIG. 1D illustrates a partially transparent perspective view of a four-catheter system and fluid paths therein.

FIG. 2A illustrates steps for splitting a catheter into a catheter hub and a catheter body.

FIG. 2B illustrates partially transparent perspective views of flushing catheter hubs.

FIG. 2C illustrates attachment regions and fastening mechanisms for interlocking catheters.

FIGS. 3A-3F illustrate various embodiments of a flushing catheter.

FIG. 4A illustrates a method for attaching a flushing catheter body retrofit.

FIGS. 4B-E illustrate various embodiments of a flushing catheter body retrofit.

FIG. 5A illustrates a method for attaching a flushing catheter extension retrofit.

FIGS. 5B-D illustrate various embodiments of a flushing catheter extension retrofit.

FIG. 6A illustrates a method for attaching a flushing hub retrofit.

FIG. 6B illustrates various embodiments of a flushing hub retrofit.

FIGS. 7A-7D illustrate examples of at least partially restrictable flush ports.

FIG. 8 illustrates various embodiments of blend space flush ports.

FIG. 9 illustrates a partial cross-section of a multi-component catheter system with a tapered flushing catheter.

### DETAILED DESCRIPTION OF THE INVENTION

The present invention may be best understood through the following detailed description and the related illustrations. In this description, like numbers refer to similar elements within various embodiments of the present invention. Within this detailed description, the claimed invention will be explained with respect to preferred embodiments. However, a person having ordinary skill in the art will readily appreciate that the methods and systems described herein are merely exemplary and that variations can be made without departing from the spirit and scope of the invention.

Some aspects of the present invention are presented as a series of steps. Any particular order of steps is merely illustrative of one possible order. It should be understood that steps may be skipped, steps may be combined, steps may be divided, and the order of the steps may be varied without departing from the spirit and scope of the invention.

### Flushing Catheter and Multi-Catheter System:

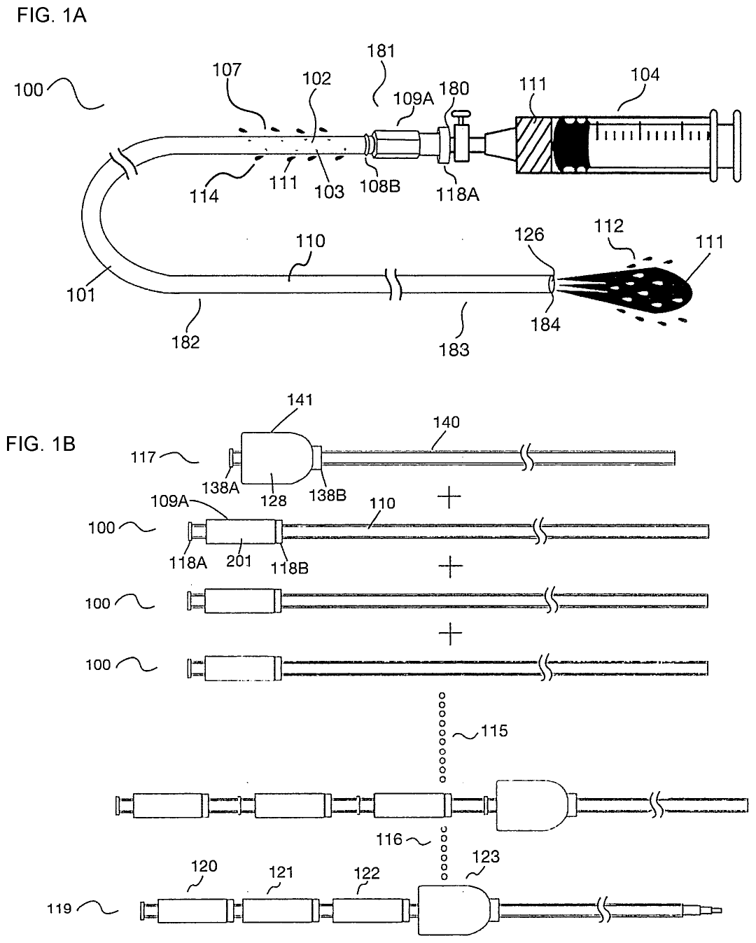

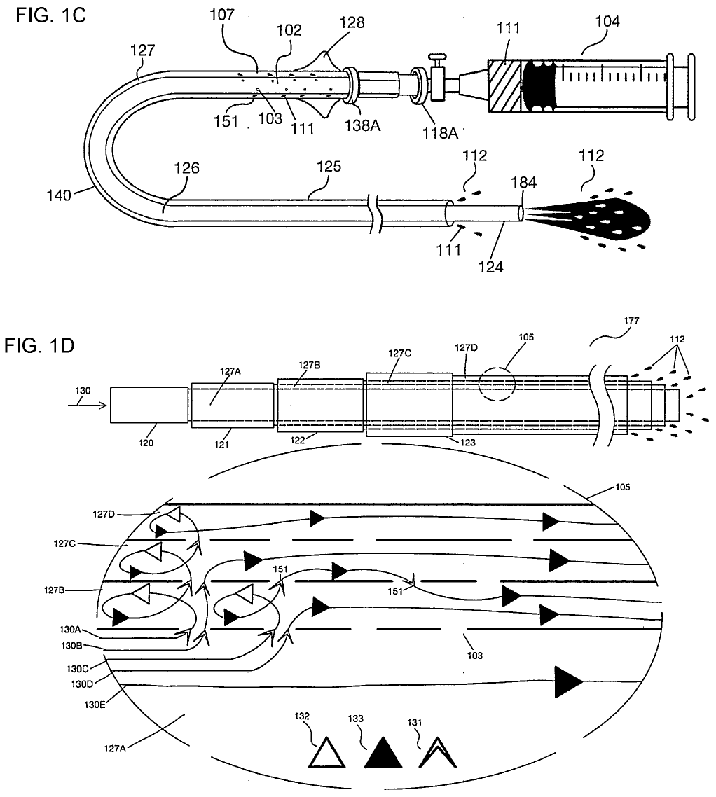

FIG. 1A shows an example embodiment of a flushing catheter 100 that includes a proximal end 180, a proximal region 181, a central region 182, a distal region 183, and a distal end 184. In this example, the flushing catheter 100 includes a cylindrical hollow tube 101 that encloses a lumen 126 that extends between the proximal end 180 and the distal end 184. The flushing catheter 100 may include a hub 109A in the proximal region 181 and a catheter body 110 in the proximal region 181, central region 182, and distal region 183. The flushing catheter 100 preferably includes at least one flush segment 102 along its length. Each flush segment 102 includes one or more flush ports 103 that perforate walls of the flushing catheter 100 and function as fluid channels. The flushing catheter 100 may include a proximal attachment region 108A, a distal attachment region 108B, a proximal fastening mechanism 118A, and/or a distal fastening mechanism 118B to connect to other catheters and to connect to fluid injection devices 104. A fluid injection device 104 may attach to the proximal end 180, engage the fastening mechanism 118A, and introduce fluid 111. The fluid 111 introduced to the flushing catheter 100 flows axially through the flushing catheter 100 to a distal end 184 to provide distal-most external fluid communication 112 and the fluid 111 flows through a partial length of the flushing catheter 100 radially through a flush port 103. Fluid 111 that flushes through a flush port 103 enters a flush region 107 that is external and adjacent to the given flush segment 102. In this example, the flush segment 102 provides proximal external fluid communication 114.

In some embodiments, the flush segments of the present invention facilitate fluid communication between many lumens of a multi-catheter system. The individual catheters of a multi-catheter system may be individually identified by at least four positional names, e.g. inner catheter, outer catheter, intermediate catheter, and adjacent catheter.

An inner catheter refers to a catheter that is nested inside at least one other catheter. In a preferred embodiment, the inner-most catheter of a multi-catheter system features a single fluid injection port. With the attachment of a single flushing device, the flush segments of the present invention enable simultaneous flushing of any number of catheters. Flush segments enable direct transluminal fluid communication between neighboring catheter lumens and indirect transluminal fluid communication between all other catheter lumens in a multi-catheter system of the present invention.

An outer catheter refers to the outermost catheter of a multi-catheter system. An outer catheter may include a sheath, a guide catheter, a reperfusion catheter, or the like. However, in some cases the outer catheter may be a flushing catheter. An outer catheter may receive direct or indirect fluid communication from an inner catheter, an adjacent catheter, or an intermediate catheter.

An intermediate catheter refers to a catheter inside of an outer catheter, e.g. the second largest catheter. In one example, an intermediate catheter is nested between an inner catheter and an outer catheter. Each catheter typically includes a male fastening mechanism, a female fastening mechanism, or both. These fastening mechanisms allow the catheters to be locked together in a sealed configuration while nested within one another. For instance, the intermediate catheter of this example will typically have a proximal female fastening mechanism attach to a smaller catheter and a distal male fastening mechanism attach to a larger catheter.

An adjacent catheter may refer to the next inner or next outer catheter in a multi-catheter system. In other words, “adjacent catheter” is context dependent and simply signifies a neighboring catheter in a multi-catheter system. An adjacent catheter may be a catheter that is nested between either two inner catheters, an inner catheter and an intermediate catheter, or an inner catheter and an outer catheter. Depending on context, an adjacent catheter may reference an outer catheter.

FIG. 1B illustrates an example construction of a four-catheter system, wherein the four-catheter system is comprised of a catheter 117 and three flushing catheters 100. In some instances, a catherization procedure may be able reduce preparation time by first interlocking several catheters according to the present invention together before purging them of air. Furthermore, providing such a multi-catheter pre-packaged in an interlocked configuration may save even more time during preparation. Simply put, a single flushing step is a more efficient use of time compared to individually flushing many devices. Thus, the present invention is especially useful during time sensitive procedures.

The first step of FIG. 1B is to assemble or select the catheters necessary to build the desired multi-catheter system. The catheter 117 may include a proximal fastening mechanism 138A, a distal fastening mechanism 138B, a lumen that extends for the full length of the catheter, a catheter hub 141, a catheter body 140, and a finger grip 128. The flushing catheters 100 may include a proximal fastening mechanism 118A, distal fastening mechanism 118B, a lumen 126 that extends for the full length of the catheter, a catheter hub 109A, a catheter body 110, a finger grip 201, and one or more flush segment(s) 102 along its length.

The second step of FIG. 1B is to nest 115 the catheters within each other in a concentric, coaxial arrangement. In this example, the smaller flushing catheters are placed within the larger flushing catheters, and all the flushing catheters are placed within the catheter 117.

The third step of FIG. 1B is to interlock 116 the individual catheters of the multi-catheter system. As a smaller catheter is advanced into a larger catheter, typically, a distal fastening mechanism of the smaller catheter engages with a proximal fastening mechanism of the larger catheter. For instance, the distal fastening mechanism 118B of a flushing catheter 100 may engage the proximal fastening mechanism 138A of the catheter 117, whereby the two fastening mechanisms seal to form a closed system between the two catheters. The triggering of the fastening mechanism may be achieved by user input in the form of axial translation and/or rotational movement.

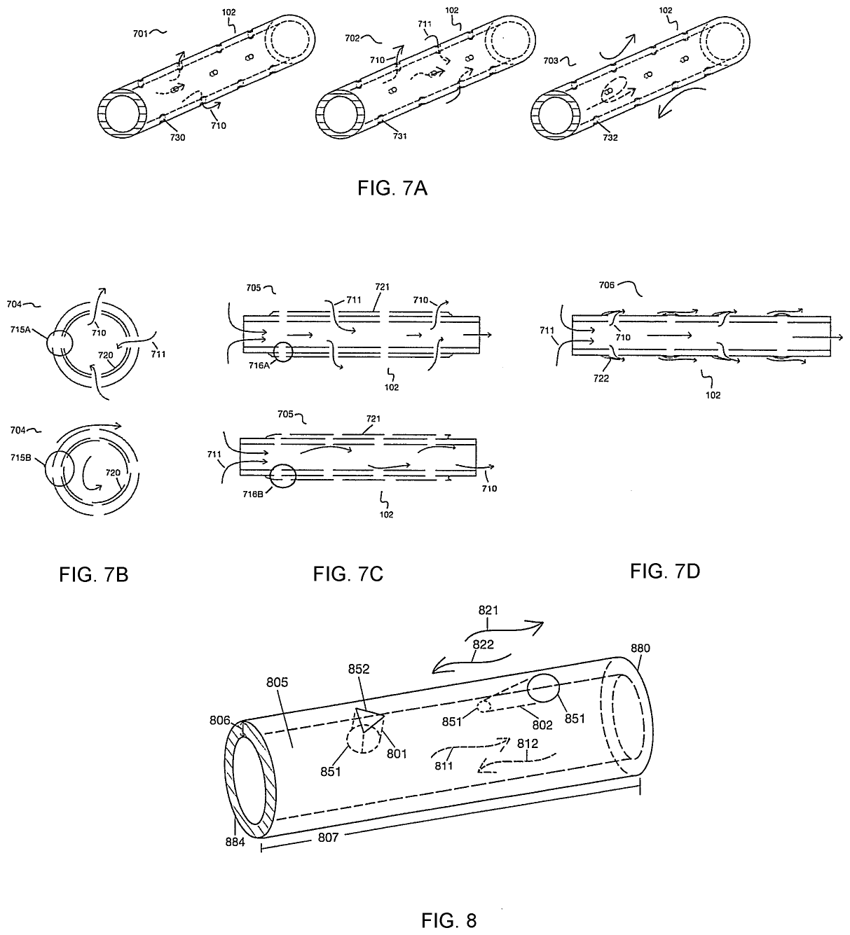

After the third step of FIG. 1B, what results is a multi-catheter system 119 in a sealed configuration. The system is sealed in that fluid access is restricted on the proximal end to the single injection port of the inner most catheter and to the open distal ends of every catheter. In a preferred embodiment, each catheter of a multi-catheter system according to the present invention includes a distal end that is open and unobstructed, wherein fluid can freely exit the distal end of each annular lumen. Once the multi-catheter system is formed, the individual catheters can be differentiated further based on their relative positions in the multi-catheter system 119. The multi-catheter system 119 of FIG. 1B is composed of an inner catheter 120, an adjacent catheter 121, an intermediate catheter 122, and an outer catheter 123. The hubs of these catheters may be differentiated with the same adjectives. In another embodiment, the outer catheter 123 is comprised of a flushing catheter 100 whose flush ports 103 are closed, a concept that will be discussed in more detail in reference to FIGS. 7A-C.

FIG. 1C illustrates an example where an inner flushing catheter 124 is nested within an outer catheter 125 to form an interlocked multi-catheter system. In this example, the inner flushing catheter 124 is a flushing catheter 100 as depicted in FIG. 1A. Fluid 111 that is introduced into the inner flushing catheter 124 may follow at least two distinct paths. The fluid 111 may flow axially along the entire length of the inner flushing catheter 124 and out the distal end 184 to ultimately provide distal-most external fluid communication 112. The fluid 111 may also flow axially through a partial length of the inner flushing catheter 124 and then radially through a flush port 103 of a flush segment 102, whereby the fluid 111 flows into a flush region 107 that is external and adjacent to the given flush segment 102, i.e. flush region fluid communication 151. In this example, the flush region 107 of flush port 102 is within the annular lumen 127 of outer catheter 125. The annular lumen 127 is the circumferential space between the outer surface of inner flushing catheter 124 and the inner surface of the outer catheter 125. In some instances, the annular lumen 127 is a narrow concentric space that does not require a large volume of fluid to be flushed. As the fluid 111 flows through the flush segment 102, the fluid first fills the flush region 107, then fills the rest of the annular lumen 127, and then exits the distal end of the outer catheter 125 to provide distal-most external fluid communication 112. The two fluid pathways of this multi-catheter system are enabled by the flush segment, which allows a single act of flushing to flush these two catheters simultaneously.

The multi-catheter system 177 of FIG. 1D is composed of an inner catheter 120, an adjacent catheter 121, an intermediate catheter 122, and an outer catheter 123. Typically, fluid access on such a multi-catheter system is restricted to a single injection port on the proximal end of the inner catheter 120. Fluid may enter the device according to arrow 130 to flush every catheter and provide distal-most external fluid communication 112, whereby all air is purged from the multi-catheter system 177. The flush segments of each catheter of the multi-catheter system 177 provide intraluminal fluid communication among central lumen 127A, adjacent annular lumen 127B, intermediate annular lumen 127C, and outer annular lumen 127D. A zoomed-in perspective of these lumens is provided by detail 105.

Referring now to detail 105 of FIG. 1D, fluid that flows through a flush port 103 may provide transluminal flow 131 (as depicted by the boomerang arrow), retrograde flow 132 (as depicted by the empty triangle), and/or principal flow 133 (as depicted by the filled triangle). Transluminal flow 131 is flow between lumens, annular lumens, or both. Retrograde flow 132 is flow that travels in a generally distal to proximal direction, i.e. backwards flow. Principal flow 133 is flow that travels in a generally proximal to distal direction, i.e. forward flow.

Detail 105 of FIG. 1D illustrates several examples of fluid paths flushing fluid may follow once the flushing fluid is injected into a multi-catheter system of the present invention. Fluid path 130A starts in central lumen 127A, has transluminal flow 131 to adjacent annular lumen 127B, loops to provide retrograde flow 132 then principal flow 133, provides additional transluminal flow 131 to intermediate annular lumen 127C, loops to provide retrograde flow 132 then principal flow 133, provides additional transluminal flow 131 to outer annular 127D, loops to provide retrograde flow 132 then principal flow 133, and eventually provides distal-most external fluid communication 112 from the outer annular lumen 127D. Fluid path 130B traverses two steps of transluminal fluid communication 131, from the central lumen 127A to the adjacent annular lumen 127B to the intermediate annular lumen 127C, and then provides principal flow 133 that eventually provides distal-most external fluid communication 112 from the intermediate annular lumen 127C. Fluid path 130C provides transluminal flow 131 in two directions, first from the adjacent annular lumen 127B to the intermediate annular lumen 127C, then from the intermediate annular lumen 127C back to the adjacent annular lumen 127B, ultimately providing distal-most external fluid communication from the adjacent annular lumen 127B. Fluid path 130D provides only one step of transluminal flow 131 before providing distal-most external fluid communication 112. Fluid path 130E may engage in one or more steps of transluminal flow 131 and/or retrograde flow 132 before ultimately providing distal-most external fluid communication from the central lumen 127A.

In some prior art designs, multi-lumen systems have an injection port for each individual lumen and annular lumen. These injection ports are usually angled (such as 45-degrees or 90-degrees) relative to the length of the multi-lumen system. These many ports encumber the proximal end of the device and add clutter and complexity. Additionally, the introduction of fluid requires an attachment step for each lumen on each injection port. Whether each injection port has its own injection device or simply a hose attached to a common injection device, the attachment of multiple components to a multi-lumen system causes clutter and encumbers the proximal end of such a system. In contrast, the flush segment(s) of the present invention enables multi-lumen access for flushing fluid with only a single attachment step to a single injection port. In a preferred embodiment, the single injection port is generally linear and aligned with a length of a flushing catheter. Additionally, the present invention enables a single step of fluid injection to flush every catheter of a multi-catheter system. Thus, the flush segment(s) and the single injection port facilitate a reduction in clutter, improves ease of use, and enables a less cumbersome multi-catheter system.

In order to flush a catheter or a multi-catheter system, the air within the system must be replaced with liquid. A catheter or multi-catheter system is flushed by injecting an “effective amount” of fluid. An effective amount can be determined through observation. In an observational approach, a user injects fluid until he or she observes fluid coming out the distal ends of all the catheters.

Flushing catheters that are nested together require less flushing fluid because there is less internal volume to be displaced. Thus, the present invention facilitates a reduction in the costs associated with flushing fluid and cuts down on unnecessary waste.

### Flushing Catheter Structure and Subcomponents:

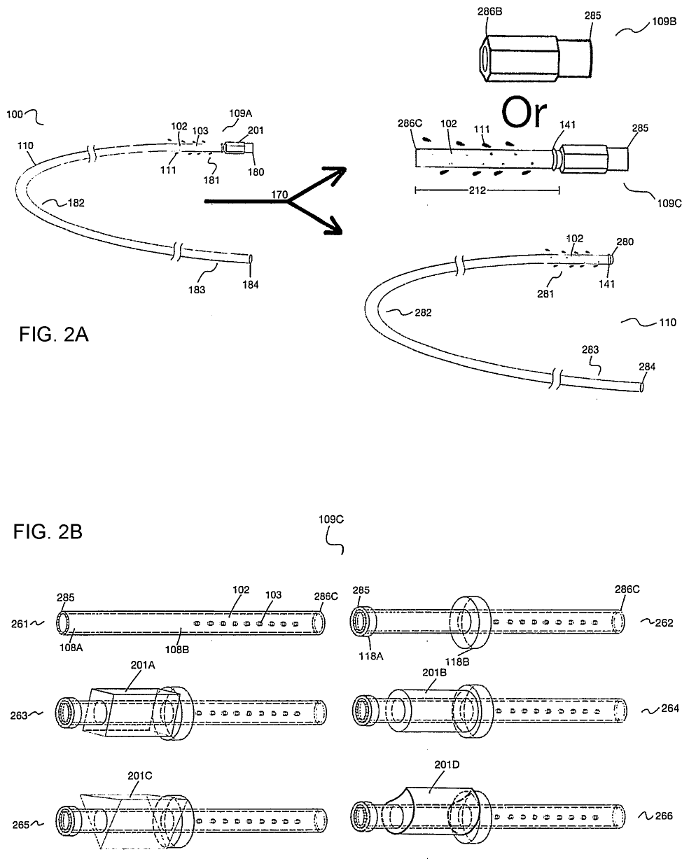

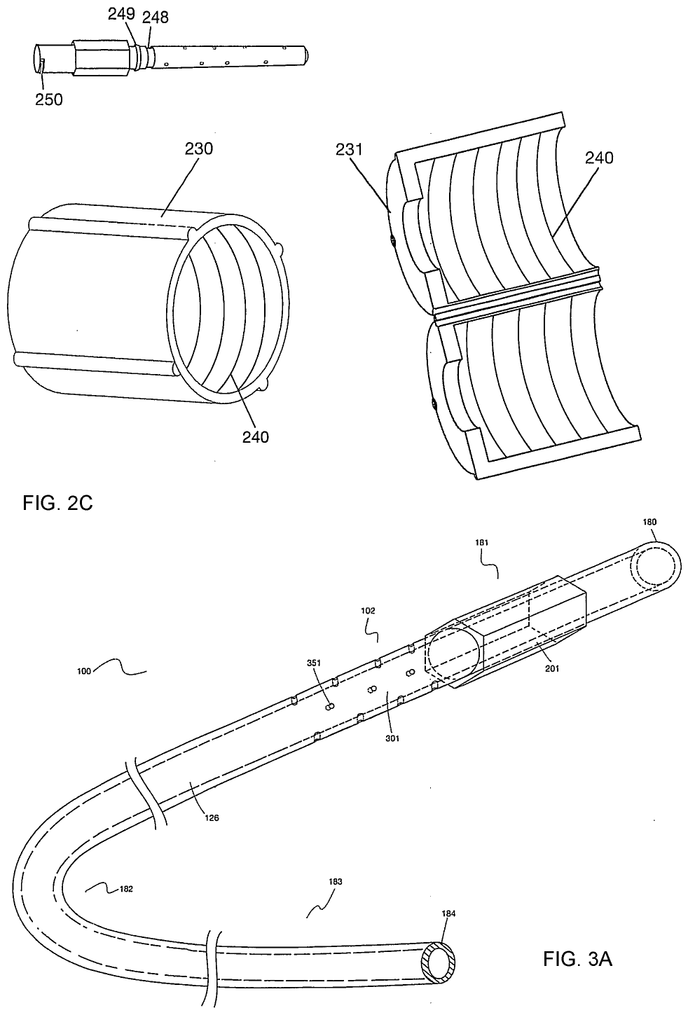

FIG. 2A shows a flushing catheter 100 and some of its key components. A flushing catheter 100 is generally comprised of a catheter hub 109A and a catheter body 110. The catheter hub 109A is configured for being held in a user's hand and the catheter body 110 is configured for entry into human vasculature. FIG. 2A illustrates an example of how a catheter hub 109A can be split 170 from a catheter body 110, whereby a single catheter yields one catheter hub (109B/109C) and one catheter body 110. A flushing catheter 100 may be separated near the optional finger grip 201 to create a short catheter hub 109B, with little or no catheter body remaining, or may be separated along the length of the catheter body 110 to create a long catheter hub 109C that includes an attached partial length of catheter body 212. The short catheter hub 109B includes a proximal end 285 and a distal end 286B. The long catheter hub 109C includes a proximal end 285, a distal end 286C, and may include one or more flush segments 102 along the partial length of catheter body 212. In some embodiments, the short catheter hub 109B is for attachment to a flushing catheter body retrofit 400, while the long catheter hub 109C is for attachment to any catheter body.

Separating or splitting a catheter or catheter component may be achieved with a blade (e.g. scissors, razorblade, etc.) or with concentrated radiative energy (e.g. laser, heat, etc.).

Once the catheter body 110 is separated from the catheter hub 109A, the catheter body 110 includes a proximal end 280, a proximal region 281, a central region 282, a distal region 283, and a distal end 284. The catheter body 110 may include one or more flush segments 102 along its length. FIG. 2A illustrates the catheter body 110 with a flush segment 102 in the proximal region 281.

FIG. 2B illustrates several variations of the long catheter hub 109C, or just “catheter hub” 109C. Such catheter hubs 109C may be manufactured as catheter hubs or may be acquired by splitting a full-length catheter according to the protocol illustrated in FIG. 2A. In a first example 261, catheter hub 109C may include a proximal end 285, a distal end 286C, a proximal attachment region 108A, a distal attachment region 108B, one or more flush segments 102, and no finger grip. In some examples, the hub includes fastening mechanisms that serve as intermediaries between two or more catheters when nested together to form a multi-catheter system. In a second example 262, catheter hub 109C may include a proximal fastening mechanism 118A and a distal fastening mechanism 118B, and no finger grip. In other examples (263, 264, 265, 266) the catheter hub 109C may include a rectangular finger grip 201A, a circular finger grip 201B, a triangular finger grip 201C, and/or a teardrop finger grip 201D, and may include attachment regions and/or fastening mechanisms. In further alternatives, the finger grip 201 may take on the shape of an oval, ellipse, square, five-or-more-sided polygon or convex polygon, star, or the like. In general, the shape is designed so that the hub portion of a flushing catheter is easy to hold and easy to manipulate. In some embodiments, a hub of a flushing catheter may include an ergonomically shaped grip. Such finger grips are positioned between the proximal end 285 and distal end 286C of catheter hub 109C.

FIG. 2C illustrates example attachment regions and fastening mechanisms that facilitate the interlocking of two or more catheters to form a multi-catheter system. The fastening mechanism may be embodied by a rotating fastening mechanism 230 with a cylindrical shape and internal threads 240. Alternatively, the fastening mechanism may be embodied by a clam shell fastening mechanism 231 that pivots along its length to unlock by opening and to lock by closing. The clam shell fastening mechanism 231 may include internal threads 240 and may include a structure that “snaps” closed as known in the art. The attachment regions (e.g. 108A/108B) may be embodied by an ident 248, a lip 249, threads 250, or a combination of such options. These attachment regions may restrict, control, or facilitate axial movement and rotational movement of a rotating fastening mechanism 230, clam shell fastening mechanism 231, or other similar fastening mechanisms as known in the art.

In any of the embodiments discussed herein, the devices may include one or more seals and/or membranes for creating a closed system. Seals and membranes allow inner catheters to pass through while also forming a seal between the inner surface of the outer device and the outer surface of the inner device to facilitate the formation of a closed system. Such seals are particularly beneficial in ensuring air does not enter devices after they have been purged of air with a fluid flush.

The flush segments 102 of a flushing catheter 100 are typically located in one or more regions among the length of the flushing catheter's catheter body 110. The flushing catheter 100 may be manufactured with one or more flush segments 102, may undergo post-processing to add one or more flush segments 102, or a flushing catheter 100 or a catheter 117 may be retrofit with a catheter subcomponent that includes one or more flush segments 102 (as will be discussed in greater detail in what follows).

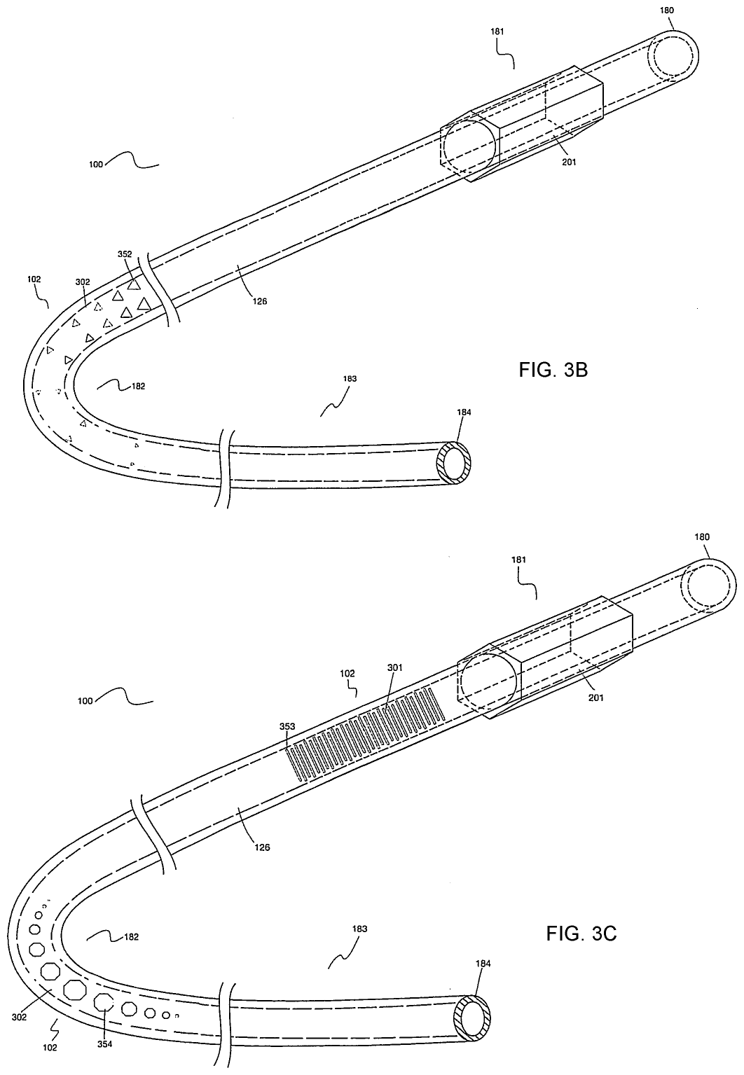

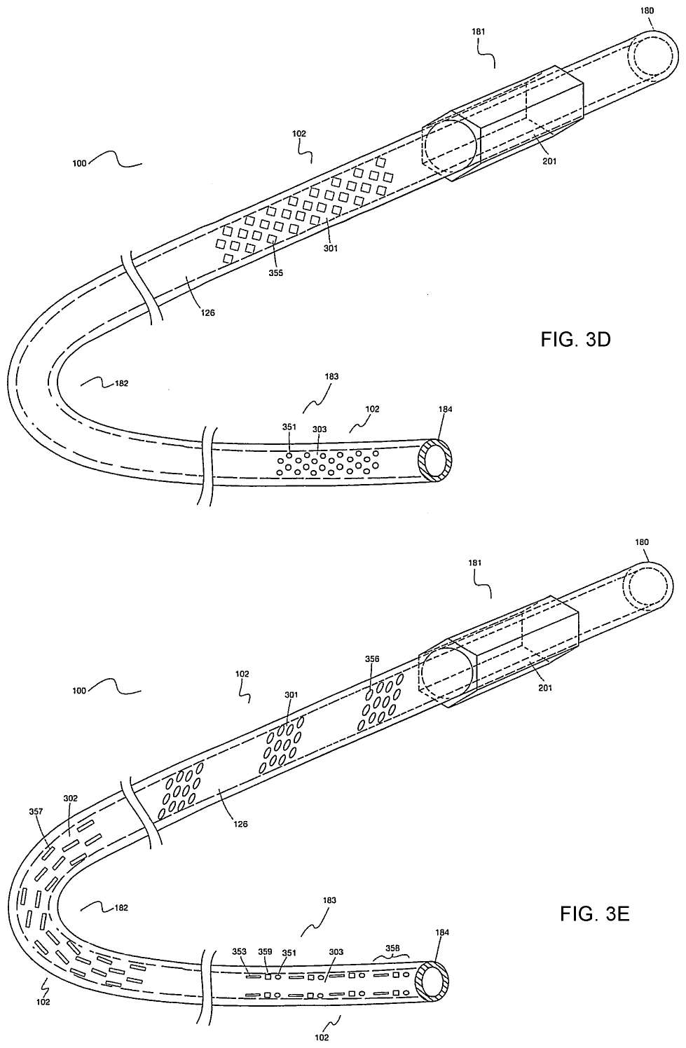

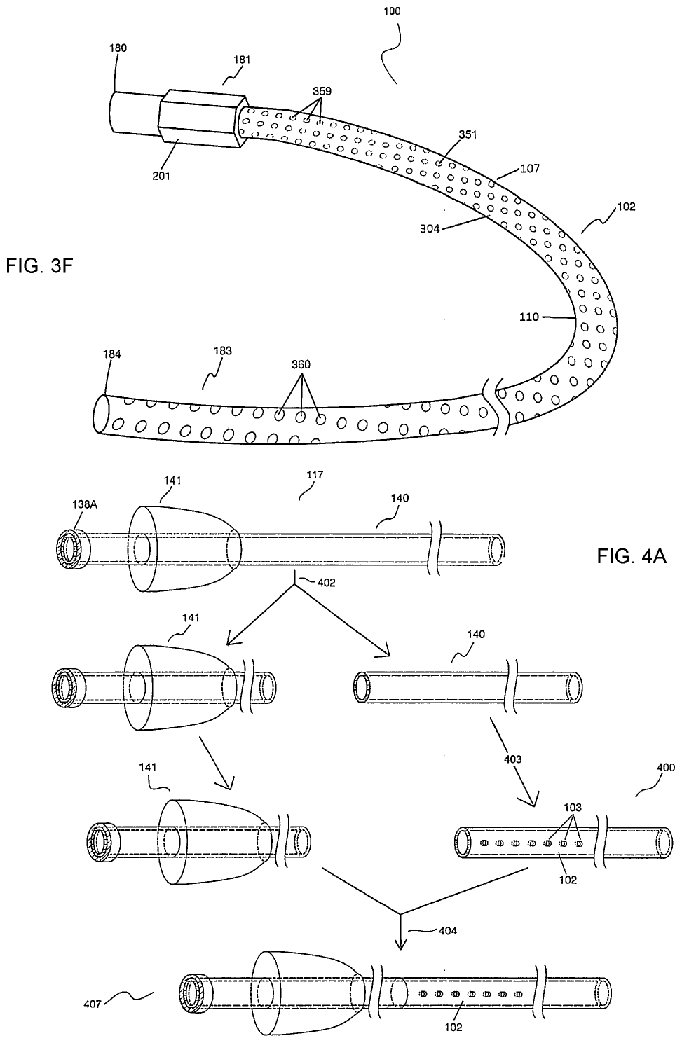

Each flush segment of the present invention features one or more flush ports. The flush ports provide fluid communication between a catheter's lumen and a flush region that is external to the catheter. In general, the flush ports may vary from one another in geometry (e.g. size, shape, pattern) and orientation along the length of a flush segment. A group of flush ports that represent a repeated pattern may be referred to as a flush sector. Flush port variability may facilitate a variable volume of fluid transfer along the length of a flush segment and thus along a flushing catheter during flushing. FIGS. 3A-5D (discussed later in more detail) illustrate numerous examples of how flush ports may vary according to the present invention.

Flush ports may vary across the length of a flush segment and/or may vary from one flush segment to another. Likewise, a catheter body may include multiple copies of one or more flush segment varieties that alternate along the length of the catheter body. The flush port trends described here may vary along the length of a flush segment according to a trend that runs in a proximal to distal direction, a distal to proximal direction, a first end to second end direction, or a second end to first end direction. For instance, a flush segment may include round flush ports that increase in size along the length of the flush segment according to one of the aforementioned directions. Flush port and flush segment variations may be smooth, gradual, and uniform in direction or these variations may be quick to change, and such trends may even, at least temporarily, reverse in direction across a short group of flush ports in the given flush segment.

Flush ports may take on many different shapes and a flush segment's flush ports may change in shape from one end of the flush segment to the other. Each flush port may take on the shape of a circle, oval, ellipse, triangle, rectangle, five-or-more-sided polygon, convex polygon, star, teardrop, or the like. In one example, a flush segment's flush ports transition from a more rectangular shape to a more square shape along the length of the flush segment. In general, a flush port's shape may vary from its neighbors by one or more dimensions, including by height, width, radius, diameter, minor axis, and/or major axis. In another example, the flush port's shape on an outer surface is different than the same flush port on the inner surface, whereby the walls or thickness of the catheter body structurally supporting the flush segment act as a blend space between the two shapes (as depicted in FIG. 8).

The orientation of a flush segment's flush ports may vary along the length of the flush segment and/or may vary between adjacent flush segments along the length of the entire catheter. Flush ports may vary in terms of spacing between adjacent flush ports. For instance, the spacing between flush ports may increase according to a trend along the length of a flush segment. Flush ports may be orientated into rows. The rows may be evenly spaced round the circumference of the given flush segment. The rows may be orientated parallel to or perpendicular to the longitudinal axis of the flush segment, or the rows may twist or tilt along such an axis. The rows may progressively vary along the length of the flush segment or may vary according to a pattern composed of multiple repeated flush sectors. In one case, the number of rows from flush segment to flush segment may increase or decrease to enable a variable volume of fluid transfer, i.e. flow rate. The phrase “flow rate” refers to a volume of fluid transfer per some increment time across a given flush port or flush segment. Flow rate refers to how much and how fast a flush port or flush segment provides fluid communication.

Variability among flush ports and flush segments may facilitate a variable flow rate along the length of the present invention. Flush port size, shape, and orientation may progressively change along the length of a flush segment to allow a greater flow rate in some regions and a lesser flow rate in other regions. A variable flow rate may progressively change along the length of a flush segment or the flow rate may follow a variable trend where flow rate increases and then decreases one or more times across the length of a flush segment. In one specific example, a flush segment has three rows of flush ports. Two rows have flush ports that increase in size in a proximal to distal direction, while the third row has flush ports that decrease in size in a proximal to distal direction, whereby the flush segment effects a variable flow rate along its length. In a further alternative, several flush segments effect a stepwise increase or decrease in flow rate along the length of a catheter.

Variability among flush ports and flush segments may facilitate a consistent flow rate along the length of the present invention. Flush port size, shape, and orientation may progressively change along the length of a flush segment to allow a consistent flow rate in one or more regions. For instance, the openings of the flush ports may grow in size slightly in a proximal to distal direction to enable a consistent flow rate for the length of the flush segment. This variability in size accounts for an injected fluid's loss of pressure head along the length of the flushing catheter. As fluid is introduced into a lumen of a catheter the pressure head is greatest near the injection site. As the fluid flows along the length of the catheter's lumen, the fluid's pressure head diminishes according to the friction of the lumen, the viscosity of the fluid, and the distance the fluid has traveled. Thus, two identical flush ports may enable different flow rates simply because one is further from the injection site than the other. Flush ports that increase in size or density in a proximal to distal direction may compensate for pressure head loss to enable a consistent flow rate across a group of flush ports.

Pressure head loss causes flush segments in a proximal region of a flushing catheter to provide a higher flow rate than identical flush segments located more distally. Additionally, fluid that flows through a flush segment in a proximal region must engage in a more limited degree of retrograde flow to fully remove air from an annular lumen as compared to flush segments located more distally. Thus, it may be preferable for a flushing catheter of the present invention to include a proximal region flush segment. A proximal region flush segment enables a higher flow rate and enables a more direct fluid path for removing air.

### Flushing Catheter Examples:

A flushing catheter according to the present invention may include one or more flush segments. Each flush segment includes one or more flush ports that may differ according to at least the variables described above. The flush ports enable fluid communication. Fluid communication typically refers to fluid that flows through a flush port from one side to the other. This fluid communication may flow from a lumen or annular lumen into a lumen, an annular lumen, a flush region, and/or an external space adjacent to a catheter. Fluid communication may also refer to intraluminal flow, transluminal flow 131, retrograde flow 132, and/or principal fluid 133 flow as described in reference to FIG. 1D. As used herein, the phrase “fluid communication” contemplates all such flows.

In order to provide the desired type of fluid communication, the flush segment(s) may be located in one or more locations along the length of the flushing catheter. For instance, a flush segment in the proximal region of a flushing catheter may provide direct and immediate proximal or proximal-most fluid communication, and may provide central, distal, and/or distal-most fluid communication indirectly, e.g. through flow that disperses from the proximal region. A flush segment in the central region of a flushing catheter may provide direct and immediate central fluid communication, and may provide proximal-most, proximal, distal, and/or distal-most fluid communication indirectly, e.g. through flow that disperses from the central region. A flush segment in a distal region of the flushing catheter may provide direct and immediate distal or distal-most fluid communication, and may provide central, proximal, and/or proximal-most fluid communication indirectly, e.g. through flow that disperses from the distal region.

In general, the terms “distal-most” and “proximal-most” refer to a subsection within a distal region or proximal region, respectively. For instance, the distal-most region refers to a more distal portion of the distal region, and the proximal-most region refers to a more proximal portion of the proximal region. In regards to fluid communication, a distal-most fluid communication may refer to fluid that exits the distal end of a catheter or out of a flush segment in a more distal portion of the distal region, i.e. distal-most region. In contrast, distal fluid communication refers to fluid that exits a flush segment in the distal region of a catheter. The same holds true for proximal and proximal-most fluid communication.

FIG. 3A illustrates an example of a flushing catheter 100. This flushing catheter 100 includes a proximal flush segment 301, i.e. a flush segment 102 located in the proximal region 181. In this example, the proximal flush segment 301 includes round flush ports 351 that are orientated into four rows that run parallel to the longitudinal axis of the flushing catheter 100. In this illustration, and those that follow, some rows may be visible while other rows are not. In this example, the rows are evenly spaced around the circumference of the flushing catheter 100, the rows are staggered, and the rows alternate between having three flush ports and four flush ports per row. In one embodiment, the flush ports are uniform in size and uniform in spacing.

FIG. 3B illustrates another example of a flushing catheter 100. This flushing catheter 100 includes a central flush segment 302, i.e. a flush segment 102 in the central region 182. In this example, the central flush segment 302 includes triangular flush ports 352 that are orientated into three evenly spaced, non-staggered rows that each include six flush ports, wherein the flush ports transition in both size and orientation along the length of the flush segment. The triangular flush ports 352 of FIG. 3B transition from large and tightly packed to small and spaced out in a proximal to distal direction, i.e. the flush ports decrease in size in a proximal to distal direction, and the space between flush ports increases in a proximal to distal direction. The central flush segment 302 of this example thereby enables a greater flow rate on a proximal side of the flush segment and lesser flow rate on a distal side.

FIG. 3C illustrates another example of a flushing catheter 100. This flushing catheter includes a proximal flush segment 301 and a central flush segment 302. In this example, the proximal flush segment 301 includes slit flush ports 353 that are orientated into one or more rows, whereby adjacent slits are staggered or offset relative to one another. In this example, the central flush segment 302 includes hexagonal flush ports 354 orientated in two rows. As illustrated in FIG. 3C, the hexagonal flush ports 354 are largest in the center of the central flush segment 302 and taper to a smaller size at both ends of the central flush segment 302. The central flush segment 302 of this example thereby enables a greater flow rate in the center of the flush segment and a progressively lesser flow rate towards the peripheral edges of the flush segment.

FIG. 3D illustrates another example of a flushing catheter 100. This flushing catheter 100 includes a proximal flush segment 301 and a distal flush segment 303, i.e. a flush segment 102 in the distal region 183. In this example, the proximal flush segment 301 includes tightly packed, offset rows of diamond flush ports 355, and the distal flush segment 303 includes tightly packed, offset rows of round flush ports 351.

FIG. 3E illustrates another example of a flushing catheter 100. This flushing catheter 100 includes a proximal flush segment 301, a central flush segment 302, and a distal flush segment 303. In further embodiments, a given region of a flushing catheter may include two or more flush segments. FIG. 3E illustrates the proximal flush segment 301 with several twisted or tilted rows of oval flush ports 356. The flush ports of this proximal flush segment 301 are orientated into bands that twist or tilt around the longitudinal axis of the catheter body. The central flush segment 302 is illustrated with several, staggered rows of rectangular flush ports 357. The distal flush segment 303 is illustrated with a flush sector 358, i.e. a repeating pattern of flush ports. This flush sector 358 includes a first slit flush port 353, a second square flush port 359, and a third round flush port 351. This distal flush segment includes three rows of flush ports, where each row includes four flush sectors.

FIG. 3F illustrates another example of a flushing catheter 100. In this example, the flushing catheter 100 includes a flush segment 102 that extends for the entire length of the catheter body 110. FIG. 3F illustrates an example of a full-length flush segment 304. The full-length flush segment 304 provides fluid communication to a flush region 107 that runs the whole length of the catheter body 110. The full-length flush segment provides fluid communication along its entire length. In a multi-catheter system, the full-length flush segment may provide fluid communication across the entire length of a larger catheter's annular lumen. As illustrated in FIG. 3F, the full-length flush segment 304 includes round flush ports 351 that increase in size in a proximal to distal direction, and the flush ports increase in spacing in a proximal to distal direction. These variations in size and spacing may enable a variable flow rate along the length of the flushing catheter 100.

In a further embodiment, the flushing catheter 100 includes a flush segment 102 that is longer than one or more regions of the catheter body 110. For instance, the flushing catheter 100 may include a flush segment along its entire length or may include a flush segment that stretches partially or completely over two or more regions. Flush segments of this type may provide partial fluid communication to some regions and complete fluid communication to other to other regions, or partial fluid communication to two or more regions.

### Flushing Catheter Retrofits:

A flushing catheter 100 may be constructed in several ways. In the examples above, the present invention included flushing catheters 100 with integrated flush ports 103. In the following examples, a flushing catheter is constructed from two or more subcomponents. In these examples, the present invention is embodied by a catheter subcomponent with at least one flush segment 102. These flushing retrofits (400, 500, 600) can be combined with catheters 117 and/or catheter parts to form a retrofitted flushing catheter (407, 507, 607). These retrofitted flushing catheters (407, 507, 607) provide at least the same fluid communication as the integrated flushing catheters described above. As used herein, “retrofit” may be used as a noun to refer to a flushing component that may be integrated into a catheter to form a flushing catheter. Additionally, “retrofit” may be used as a verb, e.g. to replace a catheter component and/or to attach a flushing subcomponent to other catheter subcomponents.

A flushing retrofit may be embodied in many ways. The flushing retrofit may include a full length of catheter body (e.g. flushing catheter body retrofit 400) or only a partial length of catheter body (e.g. flushing catheter extension retrofit 500). The flushing retrofit may include a catheter hub (e.g. flushing hub retrofit 600). Flushing retrofits may be attached to only a catheter hub, a hub and a catheter body, to only a catheter body, or to a catheter body on both sides. A flushing catheter typically requires a hub, so a flushing retrofit that does not include a hub is preferably attached to a hub or to a component that includes a hub.

In some of the examples that follow, the retrofits include a first end and a second end. The retrofit may be attached to a catheter or to catheter subcomponents on either the first end or the second end. The first end and the second end may correlate to a proximal region, a central region, or a distal region depending on the retrofit's orientation relative to the attached catheter or attached subcomponents. Proximal refers to the side of the catheter nearest the user, typically this is the side of the catheter with the hub, and distal refers to the side of the catheter furthest from the user, which is typically the end that is inserted into human vasculature during normal use.

In a multi-catheter system, a flushing retrofit may be partially covered by a neighbor catheter. For instance, the hub of a flushing retrofit will typically remain uncovered, but the proximal and central regions of the catheter body are typically covered by any neighbor catheters that are larger. The distal region may or may not be covered, depending on the relative length of the larger neighbor catheter(s). Flush segments may provide fluid communication to an external space outside of any catheter and/or an annular region within one or more other catheters, depending on their relative orientation to one another in the multi-catheter system.

### Flushing Catheter Body Retrofit Examples:

In a first set of examples, the present invention is embodied by a full-length catheter body 110 that includes one or more flush segments 102, i.e. a flushing catheter body retrofit 400. These flushing catheter body retrofits 400 may be attached to a catheter hub or to a catheter body that includes a catheter hub. In either case, once the necessary catheter subcomponents are attached to the flushing catheter body retrofit 400, a flushing catheter 407 is created.

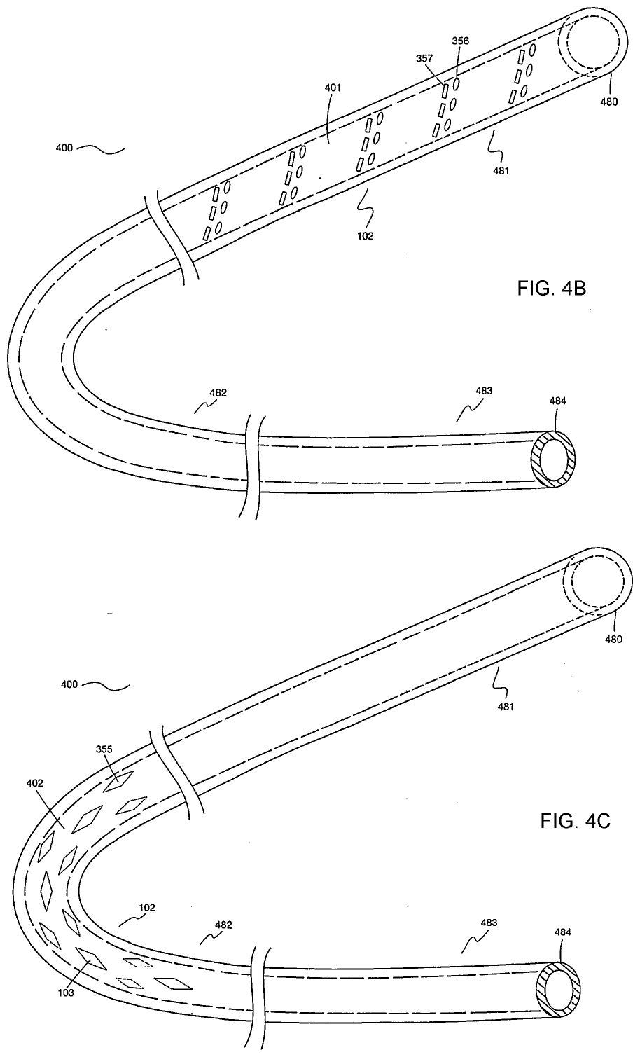

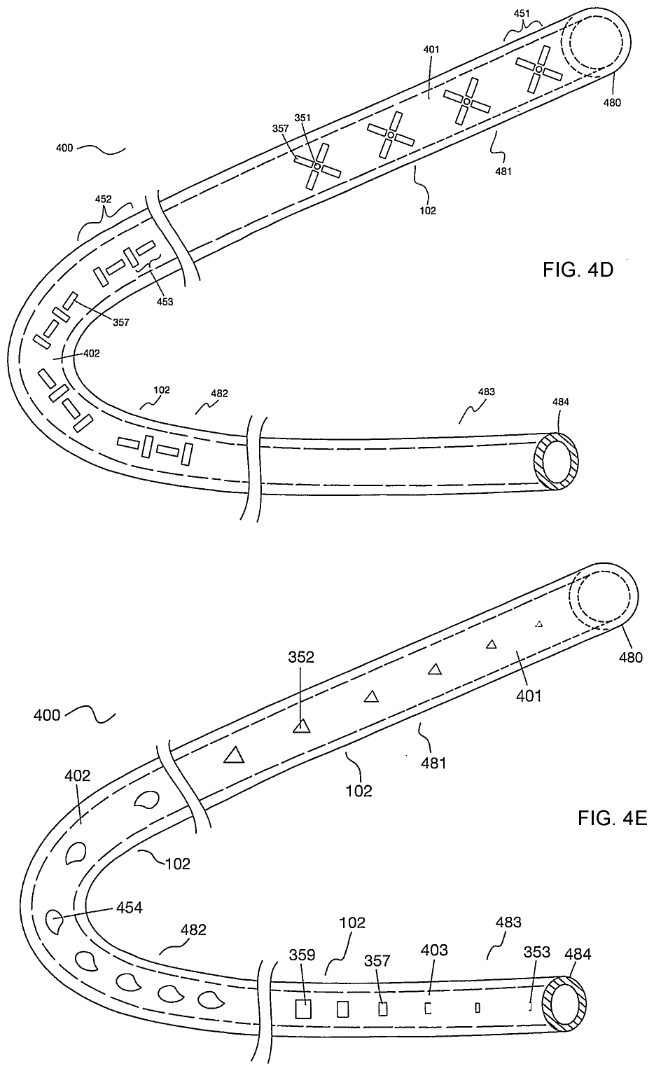

FIG. 4A illustrates an example protocol for constructing a retrofitted flushing catheter by using parts from a catheter 117 and a flushing catheter body retrofit 400, i.e. the retrofit steps. The first step is to separate 402 the catheter 117 to form a catheter hub 141 and a catheter body 140. Alternatively, a catheter hub 141 is simply provided in lieu of step one. The second step is to trade 403 the catheter body 140 for a flushing catheter body retrofit 400. The third step is to attach 404 the catheter hub 141 to the flushing catheter body retrofit 400. Once attached, a retrofitted flushing catheter, or just “flushing catheter,” 407 is formed.

FIG. 4B illustrates an example of a flushing catheter body retrofit 400. The flushing catheter body retrofit 400 includes a first end 480, a first side 481, a central region 482, a second side 483, and a second end 484. The flushing catheter body retrofit may be attached on either the first end 480 or the second end 484 to a catheter component including a catheter hub. FIG. 4B illustrates a flushing catheter body retrofit 400 with a flush segment 102 on the first side 481, i.e. a first side flush segment 401. When attached on the first end 480 the first side flush segment 401 may enable proximal fluid communication. When attached on the second end 484 the first side flush segment 401 may enable distal fluid communication. In this example, the first side flush segment 401 includes two rows of flush ports that are angled relative to the longitudinal axis of the catheter body, whereby the two rows appear to twist or tilt around the catheter body. A first row is composed of oval flush ports 356, and a second row is composed of rectangular flush ports 357.

FIG. 4C illustrates another example of a flushing catheter body retrofit 400. This example includes a flush segment 102 in the central region 482, i.e. a central flush segment 402. This central flush region 402 includes several offset rows of diamond flush ports 355. Once a flushing catheter body retrofit 400 with a central flush segment 402 is attached to the necessary catheter components, the resulting flushing catheter 407 may enable central fluid communication through its flush ports 103.

FIG. 4D illustrates another example of a flushing catheter body retrofit 400. This example includes a first side flush segment 401 and a central flush segment 402. When this example is attached on the first end 480 the first side flush segment 401 may enable proximal fluid communication. When this example is attached on the second end 484 the first side flush segment 401 may enable distal fluid communication. In either case, the central flush segment 402 may enable central fluid communication. In this example, the first side flush segment 401 includes a flush sector 451 comprised of four rectangular flush ports 357 radiating out from one round flush port 351. Such a flush sector 451 may be orientated into two rows each having four copies of the flush sector 451 along the length of the first side flush segment 401. In this example, the central flush segment 402 includes a flush sector 452 comprised of four rectangular flush ports 357 that are each rotated 90-degrees relative to one another. Such a flush sector 452 may be orientated into two rows each having four copies of the flush sector 452 along the length of the central flush segment 402. Alternatively, the central flush segment 402 may be include several copies of flush sector 453, which is comprised of two perpendicular rectangular flush ports 357.

FIG. 4E illustrates another example of a flushing catheter body retrofit 400. This example includes a first side flush segment 401, a central flush segment 402, and a second side flush segment 403. In this example, the first side flush segment 401 features triangular flush ports 352 that grow in size in a first side to second side direction. The central flush segment 402 of this example includes teardrop flush ports 454, wherein the space between subsequent flush ports decreases in a first side to second side direction. The second side flush segment 403 of FIG. 4E features square flush ports 359 on one side, slit flush ports 353 on the other side, and rectangular flush ports 357 in a middle region, whereby the flush ports gradually transition, or blend, from a more square shape to a more rectangular shape and then to a very narrow rectangle (or slit) shape, while also having increased space between subsequent flush ports, in a first side to second side direction. In this example, the flushing catheter body retrofit, once attached, forms a flushing catheter 407 capable of distal fluid communication, central fluid communication, and proximal fluid communication.

In another example, a flushing catheter body retrofit 400 may include a flush segment along its entire length or may include a flush segment that stretches partially or completely over two or more sides or regions.

### Flushing Catheter Extension Retrofit Examples:

In another set of examples, the present invention is embodied by a partial length catheter body that includes one or more flush segments, i.e. a flushing catheter extension retrofit 500. These flushing catheter extension retrofits 500 may be attached to a catheter hub and a catheter body, to only a catheter body, or to two catheter bodies. In any case, once the necessary catheter subcomponents are attached to the flushing catheter extension retrofit, a flushing catheter is created.

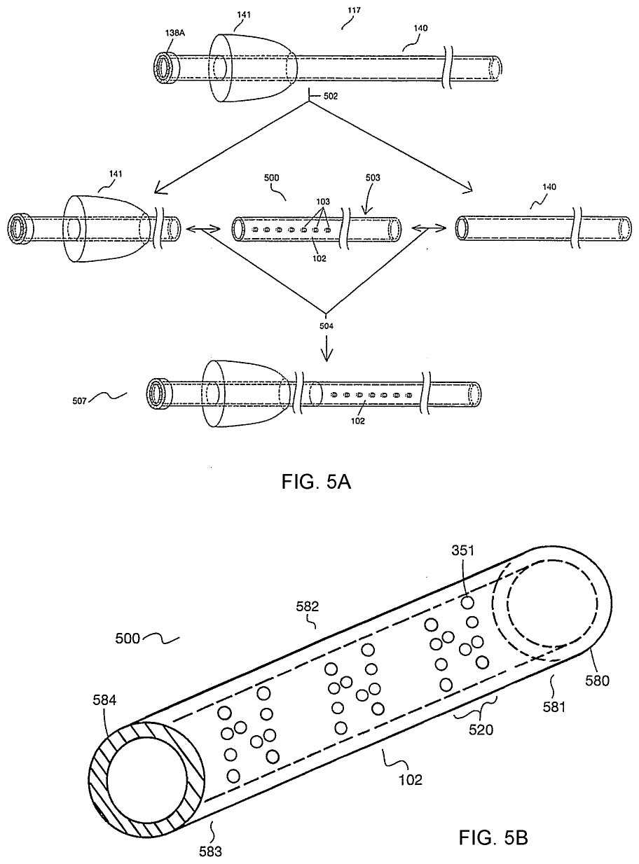

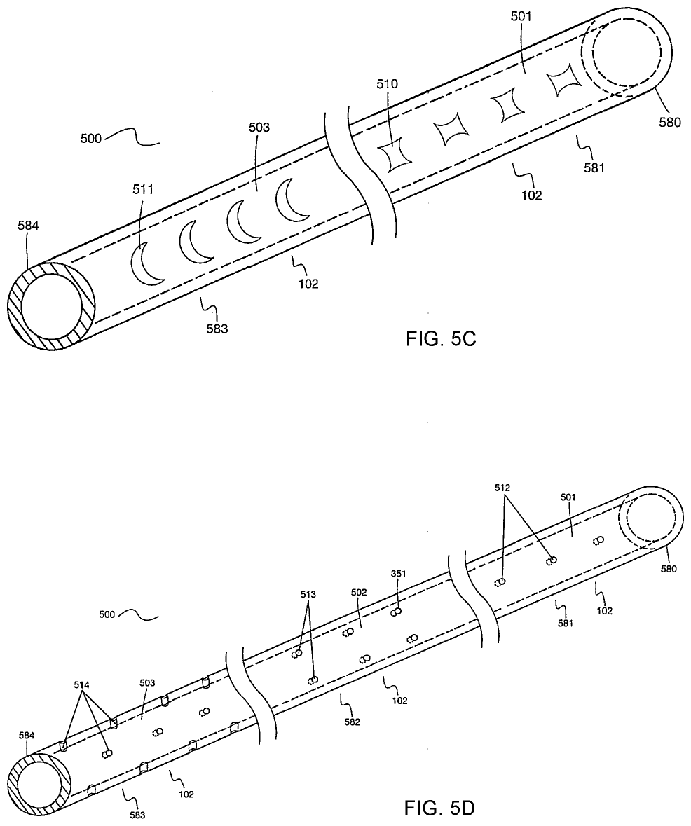

FIG. 5A illustrates an example protocol for constructing a retrofitted flushing catheter by using parts from a catheter 117 and a flushing catheter extension retrofit 500, i.e. the retrofit steps. The first step is to separate 502 the catheter 117 into a catheter hub 141 and a catheter body 140. Alternatively, a catheter hub 141 and a catheter body 140 are simply provided in lieu of step one. The second step is to position 503 a flushing catheter extension retrofit 500 between the catheter hub 141 and the catheter body 140. The third step is to attach 504 the catheter hub 141 and the catheter body 140 to the flushing catheter extension retrofit 500. Once attached, a retrofitted flushing catheter, or just “flushing catheter,” 507 is formed.

The separation step 502 introduced above may be executed in many different ways. A catheter 117 may be separated in a proximal region, a central region, or a distal region. The flushing catheter extension retrofit 500 may be attached wherever the separation is made. When the flushing catheter extension retrofit 500 is attached to a catheter 117 that was separated in the proximal region, then the retrofit may enable proximal fluid communication. When the flushing catheter extension retrofit 500 is attached to a catheter 117 that was separated in the central region, then the retrofit may enable central fluid communication. When the flushing catheter extension retrofit 500 is attached to a catheter 117 that was separated in the distal region, then the retrofit may enable distal fluid communication. In some instances, the flushing catheter extension retrofit 500 has a length sufficient to extend at least partially through two or more regions to provide a fluid communication across two or more regions. In an alternative construction, a catheter hub 141 and a catheter body 140 are simply provided in lieu of the separation step. Such components may have the same variable sizes as those produced by the variable separation steps detailed above. Thus, the present invention contemplates enabling proximal, central, distal fluid communication, and/or a combination of such fluid communications according to this alternative construction method as well.

FIG. 5B illustrates an example of a flushing catheter extension retrofit 500. In this example, a flush segment 102 extends for the entire length of the flushing catheter extension retrofit 500. This flush segment 102 includes flush sector 520 comprised of round flush segments 351 orientated in a zig-zag pattern. The zig-zag flush sector 520 repeats several times along the length of the flushing catheter extension retrofit 500 shown in FIG. 5B. The flushing catheter extension retrofit 500 includes a first end 580, a first side 581, a central region 582, a second side 583, and a second end 584.

FIG. 5C illustrates an example of a flushing catheter extension retrofit 500. This example includes two distinct flush segments 102. A first side flush segment 501 in the first side 581 and a second side flush segment 503 in the second side 583. This first side flush segment 501 includes concave polygon flush ports 510 and this second side flush segment 503 includes crescent flush ports 511. The flow rate of the flush ports of this example may be greater with internal fluid flowing in one direction as compared to the other, due to the geometry of the flush port's openings, as detailed more thoroughly in reference to FIG. 8.

FIG. 5D illustrates an example of a flushing catheter extension retrofit 500. This example includes three distinct flush segments 102. A first side flush segment 501, a central flush segment 502, and a second side flush segment 503. The first side flush segment 501 includes two rows 512 of round flush ports 351. The central flush segment 502 includes three rows 513 of round flush ports 351. The second side flush segment 503 includes four rows 514 of round flush ports 351. If this flushing catheter extension retrofit 500 is attached with the first end 580 orientated closest to a catheter hub, then the variable amount of flush ports rows from flush segment to flush segment of this flushing catheter extension retrofit 500 will enable a step-wise increase in flow rate in a proximal to distal direction. If this flushing catheter extension retrofit 500 is attached with the second end 584 orientated closest to a catheter hub, then the variable amount of flush ports rows from flush segment to flush segment of this flushing catheter extension retrofit 500 will enable a step-wise decrease in flow rate in a proximal to distal direction.

### Flushing Catheter Hub Retrofit Examples:

In another set of examples, the present invention is embodied by a catheter hub with a partial length of catheter body, wherein the partial length of catheter body includes one or more flush segments, i.e. a flushing hub retrofit 600. These flushing hub retrofits may be attached to a catheter body. Such hubs typically include a proximal end 285 and a distal end (286B/286C). Once attached to the necessary catheter subcomponents, a flushing catheter 607 is created.

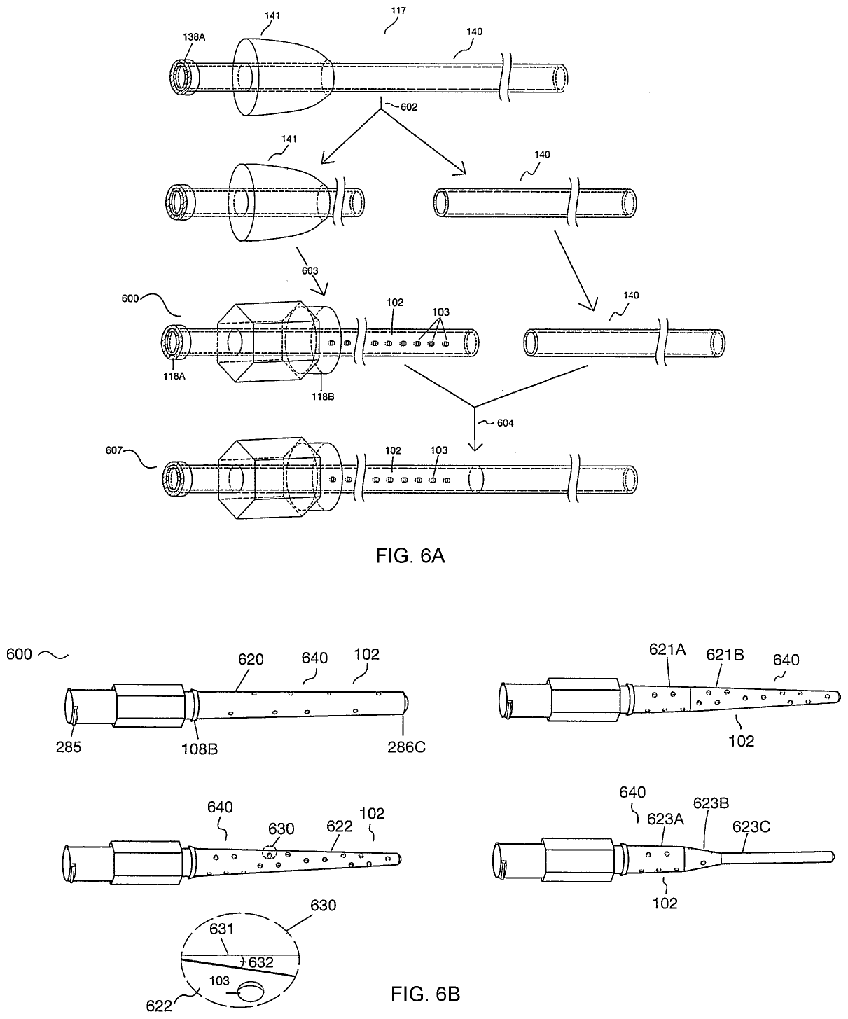

FIG. 6A illustrates an example protocol for constructing a retrofitted flushing catheter by using parts from a catheter 117 and a flushing hub retrofit 600, i.e. the retrofit steps. The first step is to separate 602 the catheter 117 into a catheter hub 141 and a catheter body 140. Alternatively, a catheter body 140 is simply provided in lieu of step one. The second step is to trade 603 the catheter hub 141 for the flushing hub retrofit 600 by placing it adjacent to the catheter body 140. The third step is to attach 604 the catheter body 140 to the flushing hub retrofit 600. Once attached, a retrofitted flushing catheter, or just “flushing catheter,” 607 is formed.

FIG. 6B illustrates several embodiments of a flushing hub retrofit 600. The flushing hub retrofit 600 is generally of the long catheter hub 109C variety so there is adequate room for a flush segment 102 on the partial length of catheter body 640. However, in some instances, a short catheter hub 109B may be used to form a flushing hub retrofit 600 and/or flushing catheter 607. The partial length of catheter body 640 may come in a variety of geometries. In general, the partial length of catheter body can be conceptually split into three regions. A proximal region is closest to the optional finger grip, a central region is in the center of the partial length of catheter body, and a distal region is the region furthest from the optional finger grip.

In one example, the catheter body 640 of the flushing hub retrofit 600 is entirely straight, maintaining an identical inner diameter and outer diameter from end to end. In a first illustrated example, the flushing hub retrofit 600 has a relatively linear catheter body 620. In alternative embodiments, the flushing hub retrofit's 600 catheter body may transition from a relatively large proximal diameter to a relatively small distal diameter. Such transitions in diameter may be smooth and gradual or the transition may occur over one or more steps. In a second illustrated example, the flushing hub retrofit 600 has a catheter body with a first taper 621A, that is relatively shallow, and then steps down to a second taper 621B, that is relatively steep. In a third illustrated example, the flushing hub retrofit 600 includes an angled catheter body 622, which is enlarged by detail 630. Angled catheter body 622 has an acute angle 632 relative to horizontal axis 631. When an angle for flushing hub retrofit's catheter body is referenced, it is understood to be the angle between a horizontal axis and the catheter body as depicted in detail 630. In some instances, a particular angle may beneficially provide a greater flow rate across the flush ports that have faces at the chosen acute angle. Alternatively, an angle may be chosen that reduces the flow rate. The angle may range between 1 degree and 45 degrees. In a fourth illustrated example, the flushing hub retrofit 600 includes a catheter body with a first angle 623A in a proximal region that is relatively shallow, then steps to second angle 623B in a central region that is relatively steep, and then steps again to a third angle 623C in a distal region that is relatively shallow. In one specific example, a flushing hub retrofit 600 has a first angle in the range of 5°-10°, a second angle in the range of 15°-25°, and a third angle in the range of 0°-5°. Of course, these angles are only exemplary and other angles consistent with the more general descriptions of these various embodiments are contemplated as within the scope of the present invention. For instance, in other embodiments, the outer diameter may oscillate one or more times between growing and shrinking in a proximal to distal direction according to a variety of favorable angles.

### Restrictable Flush Ports:

In any of the embodiments discussed herein, the flush ports may be at least semi-restrictive over certain types of fluid flow. Flow restriction may be achieved with a restriction means, such as valves, and pressure responsive slits. Such restriction means are capable of selectively restricting flow across individual flush ports. For instance, reverse Tuohy seals may manipulate the size of an individual flush port's opening. A pressure responsive slit may open and close under particular pressure differentials, such as when the pressure within the catheter is greater than the pressure outside of the catheter.

Flow restriction may also be achieved with covers for individual flush ports or covers for entire flush segments. Covers for individual flush ports may be embodied by flaps or hatches that are capable of selectively restricting fluid access to an individual flush port. For instance, a flap on the outer surface of a catheters flush port may open when fluid pressure inside is greater than outside pressure and may remain closed when fluid pressure inside is lower than outside pressure. Such a flap may be configured to enable one-way flow only. Covers for entire flush segments may be embodied by thin tubes, sheaths, or liners that are capable of selectively restricting access across a group of flush ports. An outer sheath or an inner liner with flush port sized holes may be axially translated and/or rotationally translated via a cord or wire mechanism controllable at the hub to move the sheath's or liner's holes at least partially out of alignment with the flushing catheter's flush port holes. In other embodiments, a structure within the walls of the flushing catheter may axially and/or rotationally translated (e.g. like a moonroof) to at least partially obstruct the flow of fluid through a set of flush ports.

In general, fluid flow may be restricted through an automated mechanism or through user control. Fluid flow may be automatically restricted with sensor controlled flush ports or by mechanical design. Fluid flow may be manually restricted with user controls such as slides, switches, and knobs. For instance, a slide may close two-way flush ports and one-way flush ports. A switch may restrict a two-way flush port to only allow fluid flow in one direction. A knob may be twisted to modulate or partially restrict fluid flow across one or more flush ports, whereby the degree the knob is twisted corresponds to the degree fluid flow is restricted. Such control features may be readily implemented by those with skill in the art.

FIG. 7A illustrates several examples of how flush ports may be at least semi-restrictive to some types of flow. In a first illustrated example, a flush segment 701 includes one-way flush ports 730 that only allow fluid to flow in one direction, which, in this case, is from inside to outside according, i.e. exit flow 710. Alternatively, the one-way flush ports may allow fluid flow in the opposite direction instead. In a second illustrated example, a flush segment 702 includes two-way flush ports 731 that allows exit flow 710 and entry flow 711, which indicates flow from outside to inside. In a third illustrated example, a flush segment 703 includes fully restricted flush ports 732 that restrict fluid flow in both directions.

FIG. 7B illustrates a radial cross-section of an inner moveable sheath 720 configured for rotational movement. Inner movable sheath 720 may include includes holes with the same geometries as the flush ports of the flush segment. In a first orientation, the holes of the inner movable sheath 720 are aligned with the individual flush ports as shown in detail 715A, whereby the inner movable sheath 720 may enable exit flow 710 and entry flow 711 across the aligned flush ports. In a second orientation the holes are unaligned with the individual flush ports as shown in detail 715B, whereby the inner movable sheath 720 is at least semi-restrictive of fluid flow. In this example, inner movable sheath 720 rotates to transition between an open and closed orientation. In further alternatives, the inner movable sheath 720 may axially translate to transition between an open and closed orientation.

FIG. 7C illustrates an outer movable sheath 721 configured for axially movement. Outer movable sheath 721 may include holes with the same geometries as the flush ports of a flush segment. In a first orientation, the holes of the outer movable sheath 721 are aligned with the individual flush ports as shown in detail 716A, whereby the outer movable sheath 721 may enable exit flow 710 and entry flow 711 across the aligned flush ports. In a second orientation the holes are unaligned with the individual flush ports as shown in detail 716B, whereby the outer movable sheath 721 is at least semi-restrictive of fluid flow. In this example, outer movable sheath 721 axially translates to transition between an open and closed orientation. In further alternatives, the outer movable sheath 721 may rotate to transition between an open and closed orientation.

FIG. 7D illustrates a flush segment that includes hatches 722 positioned on the outer surface of the catheter body that allows exit flow 710 and restricts entry flow 711. In further alternatives, the hatches 722 are located within the lumen of the catheter on the inner surface adjacent to a flush segment, whereby the hatches 722 are one-way valves in the opposite direction, e.g. allows entry flow 711 and restricts exit flow 710.

In one embodiment of the present invention, two or more catheters are interlocked to form a multi-catheter system. The outer most catheter includes a flush segment 102 with flush ports 103 that are selectably restrictable by any of the methods described above. The inner catheters include at least one flush segment 102 and may or may not include a mechanism for selectably restricting flow across their flush ports. In such an embodiment, flushing may be restricted to certain catheter lumens and exclude others. Another option is to close at least a portion of the flush ports after the system has been purged of air with a fluid flush.

In one example, a flushing catheter includes at least one flush segment with selectably restrictable flush ports. When this example is used with an aspiration source, the flush ports may be opened and closed to manipulate the pressure within the flushing catheter.

FIG. 8 illustrates some examples of blend space flush ports. A standard flush port is the same shape, size, and orientation on both the outer surface opening and the inner surface opening of the given flush segment. Additionally, standard flush ports perforate a hole through a thickness 806 of the catheter body that is orientated perpendicular to the longitudinal axis of the catheter body. Blend space flush ports may have an opening on the outer surface that differs from the opening on the inner surface according to shape, size, and orientation. In a first example, a blend space flush port 801 features an outer surface opening 852 that is triangular in shape and an inner surface opening 851 that is circular in shape. The thickness 806 of the catheter body provides a transition length over which the shape of the blend space flush port 801 progressively transitions from a triangular shape to a circular shape.

In a second example of FIG. 8, blend space flush port 802 has an outer surface opening that differs from the inner surface opening in terms of size and orientation. The outer surface opening is larger and is closer to the first end 880. The inner surface opening is smaller and closer to the second end 884. Blend space flush port 802 has not only the width 806 of the catheter body between the inner and outer openings, but also includes a portion of the length 807 of catheter body between the two openings. Together, the width 806 and the portion of catheter body length provide a material through which the blend space flush port 802 can blend between the different size and orientations of the two openings, which, in this example, forms an angled, conic shaped blend space for the flush port. The variable size and non-perpendicular, or offset, nature of the blend space flush port 802 may facilitate some types of transluminal flow while being restrictive to other types of transluminal fluid flow. For instance, fluid flowing in the direction of the flush port's angle (822/811) may more readily flow through the blend space flush port 802, while fluid flowing against the angle of the flush port (812/821) may be partially restricted from crossing the blend space flush port 802. In general, fluid flowing “with the angle” refers to fluid whose flow path must only make an acute angle to traverse the flush port. Additionally, fluid flowing on the side of the larger opening (821/822) may more readily enter the flush port than fluid flowing on the side of the smaller opening (811/812). In this way, blend space flush port 802 is an angled flush port that is preferential to some directions of fluid flow and at least slightly restrictive of other directions of fluid flow.

Although FIG. 8 illustrates only isolated blend space flush ports, it should be appreciated that a flush segment and a flush sector according to the present invention may be comprised of numerous blend space flush ports. The many blend space flush ports may differ according to at least all the variables of the standard flush ports discussed earlier.

### Distal Taper:

To reach a treatment site with a particular intravascular device, it is common practice to use several coaxial components in concert, such as access catheters, guide catheters, reperfusion catheters, microcatheters, guidewires, and other similar devices. Usually, a guidewire is the first device to fully navigate the vasculature and arrive at the treatment site. The guidewire then serves as a rail that guides other devices to the treatment site. However, as other devices track over the guidewire, they may unintentionally snag on branch vessels, which may cause vasculature damage and/or halt progression. This risk increases proportionally with the difference in diameter between the rail guide and the tracked device.

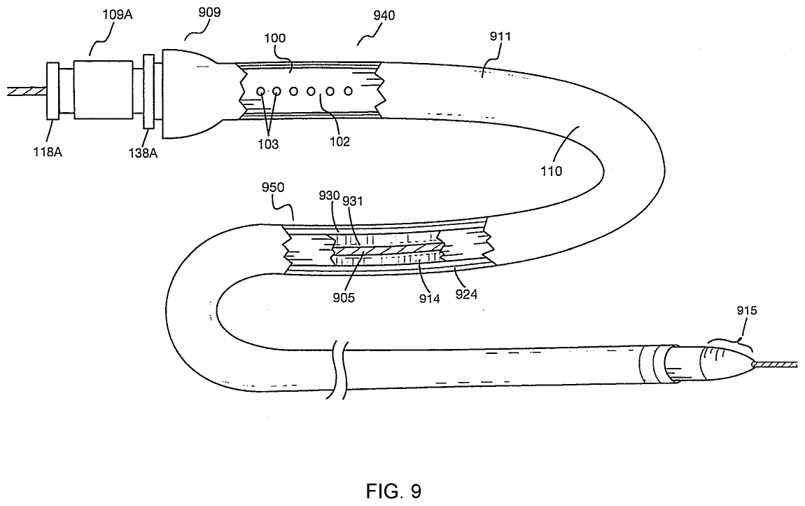

FIG. 9 illustrates a partially transparent view of a three-device system that includes an outer catheter 911, an inner flushing catheter 100 with an optional tapered distal end 915, and a guidewire 905 all situated in a coaxial relationship. The tapered distal end 915 substantially reduces the risk of snagging on vasculature protrusions by reducing or eliminating the gap between a guidewire and a catheter. The taper of the flushing catheter 100 ideally begins a short distance after the distal end of the catheter 911 and extends distally beyond the catheter. In a preferred embodiment, the taper starts after the distal end of catheter 911 even while traversing exceptionally tortuous vasculature. In some cases, the hub 109A of the flushing catheter is fastened to the hub 909 of the outer catheter 911 according to the means described previously. In a first cut through 940 the outer catheter 911 is cut away and a flush segment 102 and its flush ports 103 are visible on the inner flushing catheter 100. In a second cut through 950 the outer catheter 911 is cut away, the inner flushing catheter 100 is partially cut away, and the guidewire 905 is visible. An inner annular lumen 931 is visible between an outer surface of the guide wire 905 and an inner surface of the flushing catheter 100. An adjacent annular lumen 930 is visible between an outer surface of the flushing catheter 100 and an inner surface of the catheter 911. In this example, the flushing catheter 100 includes optionally thick walls 914, while the catheter 911 has thin walls 924. In one example, the walls of the flushing catheter 100 have a thickness between 0.010-0.050 in. These thick walls 914 help fill the volume between the outer surface of the guidewire 905 and the inner surface of the catheter 911. With this volume filled, a lesser volume of flushing fluid is required to purge both the catheters of air. In some cases, the flushing catheter's 100 outer diameter matches (within 0.005 in) an inner diameter of an outer catheter, and the flushing catheter's inner diameter matches (within 0.005 in) an outer diameter of a guidewire.

In one example, the present invention is embodied by method for modifying a catheter, wherein the method includes a step of selecting a catheter from an inventory of pre-fabricated catheters, said selected catheter comprising an elongate catheter body having a proximal region, a central region, a distal region, and a central lumen extending therethrough and a hub connected to the proximal region of the catheter body, said hub having a single injection port which provides a sole connection to a proximal end of the central lumen. Such a method may also include a step of forming flush ports in at least one of the proximal region, the central region, and the distal region, wherein the flush ports allow radial fluid flow through a wall of the elongate catheter body. Such a method may further include a step of introducing holes in an inner or outer sheath that match the holes made in the flushing catheter and affixing the sheath to the flushing catheter, wherein the sheath is configured to either axially translate, rotationally translate, or both.

In another example the present invention is embodied by a method for fabricating a flushing catheter, the steps including: (1) selecting a catheter hub or a catheter body; (2) selecting a flush retrofit from among a flushing catheter body retrofit, a flushing catheter extension retrofit, or a flushing hub retrofit; and (3) attaching the flush retrofit to the either the catheter hub, the catheter body, or both.

Any of the embodiments discussed herein may be constructed from one or more materials. For instance, the components may be constructed from a polymer such as: silicon, polyurethane, polyvinyl chloride, Nylons, or polyether block amides. Alternatively, the components may be constructed from an alloy such as: stainless steel, platinum, tungsten, and NiTinol. In some embodiments, the components may utilize a combination of different polymers and alloys. In some embodiments, some components of a given retrofit may be polymer based and other components may be alloy based. In one example, a retrofit is formed from hardened plastic with punch holes for flush ports. In another example, a retrofit is formed from an alloy based hypotube and the flush ports are cut into the hypotube.

In any of the embodiments discussed herein the retrofit may be fixedly attached by one or more methods. A catheter body, a hub, or both may be attached to a retrofit with an adhesive (e.g. UV glue), with a polymer jacket overmold, with a weld between the components, with heat induced melting, with a friction joint, with a fixed coupler, with a rotating connector, with a snap fit mechanism, with a clamp mechanism, or any combination of aforementioned methods. In some embodiments, a fastening mechanism may be first fixedly attached to a catheter body, a hub, or both, which then latches onto a structure of the given retrofit. Alternatively, the fastening mechanism is first attached to one or more sides of the retrofit before it is fitted onto other components.

While a number of preferred embodiments of the invention and variations thereof have been described in detail, other modifications and methods of using and medical applications for the same will be apparent to those of skill in the art. Accordingly, it should be understood that various applications, modifications, and substitutions may be made of equivalents without departing from the spirit of the invention or the scope of the claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A flushing catheter, comprising:
an elongate catheter body having a proximal region, a central region, a distal region, and a central lumen extending therethrough;
a hub connected to the proximal region of the catheter body, said hub having a single injection port which provides a sole connection to a proximal end of the central lumen; and
a flush segment having a length and one or more flush ports, wherein the flush segment is located along the length of the catheter body;
wherein the elongate catheter body is configured to coaxially insert into one or more additional catheters so that a flushing fluid entering the central lumen of the elongate catheter body through the single injection port will flow radially outwardly through the one or more flush ports to enter an annular lumen formed between an outer surface of the elongate catheter body and an inner surface of an outermost additional catheter.

2. The flushing catheter of claim 1, wherein the central lumen has an open distal end so that flushing fluid entering the central lumen of the elongate catheter body through the single injection port flows axially and outwardly through the open distal end as well as flowing radially and outwardly through the one or more flush ports.

3. The flushing catheter of claim 1, wherein the distal region includes a tapered distal end.

4. The flushing catheter of claim 1, wherein the elongate catheter body has walls that fill at least 70% of the volume between an outermost catheter and an inner guidewire, in at least the distal region.

5. The flush ports of claim 1, wherein the flush ports have an outside opening and an inside opening and the outside opening is a different shape than the inside opening, wherein a thickness of the elongate catheter body provides a distance over which the inside opening progressively blends into the shape of the outside opening.

6. The flushing catheter of claim 1, wherein the flush segment is located in the proximal region of the flush catheter.

7. The flushing catheter of claim 1, wherein the flush segment is located in the central region of the flush catheter.

8. The flushing catheter of claim 1, wherein the flush segment is located in the distal region of the flush catheter.

9. The flushing catheter of claim 1, wherein the flush segment at least partially extends over two or more of the proximal region, the central region, and the distal region.

10. The flushing catheter of claim 1, wherein the flushing catheter includes two or more flush segments.

11. The flushing catheter of claim 1, wherein the proximal region includes one or more fastening mechanisms configured to interlock with other catheters and other devices.

12. The flushing catheter of claim 1, wherein the proximal region includes a first fastening mechanism for interlocking to a larger catheter and second fastening mechanism for interlocking to a smaller catheter, whereby interlocked smaller catheters seal the proximal end of larger catheters.

13. The flushing catheter of claim 1, wherein the flush ports are configured to enable transluminal fluid communication between the lumen of the flushing catheter and an annular lumen of an adjacent catheter, whereby fluid communication to the annular lumen flushes all air from the adjacent catheter.

14. The flush ports of claim 1, wherein the flush ports have a round or polygon shape.

15. The flush ports of claim 1, wherein the flush ports transition from a first shape to a second, different shape along the length of the flush segment.

16. The flush ports of claim 1, wherein the flush ports progressively increase in size across the length of the flush segment, whereby the flush ports enable a greater flow rate on one side of the flush segment.

17. The flush ports of claim 1, wherein the flush ports are spaced increasingly far apart along the length of the flush segment, whereby the flush ports enable a greater flow rate on one side of the flush segment.

18. The flush ports of claim 1, wherein the flush ports are angled ports that enable a greater flow rate for fluid flowing with said angle.

19. The flushing catheter of claim 1, including a cover for the one or more flush ports, wherein the cover is configured to at least partially restrict fluid flow across the flush ports.

20. The flushing catheter of 19, wherein the cover includes holes matching the geometry of the one or more flush ports and the cover is configured to axially translate, rotationally translate, or both, said translation causing the holes to move out of alignment with the one or more flush ports to at least partially restrict flow.

21. The flushing catheter of claim 1, wherein the flush segment is situated within a lumen of an adjacent catheter, whereby the flush segment provides intraluminal fluid communication to an annular lumen of the adjacent catheter.

22. The flushing catheter of claim 1, wherein the flush segment is located on the hub.

23. The flushing catheter of claim 22, wherein the hub includes a finger grip and the flush segment is located distally of said finger grip.

24. The flushing catheter of claim 1, wherein the hub includes a distal end that is configured for attachment to a proximal end of the elongate catheter body.

25. The flushing catheter of claim 1, wherein an outer diameter of the flushing segment tapers from a larger proximal diameter to a smaller distal diameter.

26. The flushing segment of claim 25, wherein the taper is progressive.

27. The flushing segment of claim 25, wherein the taper occurs over a series of steps and a first step has a lesser taper and a second step has a greater taper.

28. A multi-catheter system, comprising:
an inner flushing catheter and an adjacent catheter;
wherein the inner flushing catheter comprises:
an elongate inner catheter body having a proximal region, a central region, a distal region, and a central lumen extending therethrough;
an inner hub connected to the proximal region of the elongate inner catheter body, said hub having a single injection port which provides a sole connection to a proximal end of the central lumen; and
a flush segment having a length and one or more flush ports, wherein the flush segment is located along the length of the catheter body;
wherein the adjacent catheter comprises:
an elongate adjacent catheter body having a proximal region, a central region, a distal region, and a central lumen extending therethrough; and
an adjacent hub connected to the proximal region of the adjacent catheter body;
wherein the elongate inner catheter body is configured to coaxially insert into the adjacent catheter and adjacent hub to form an annular lumen between an outer surface of the elongate inner catheter body and an inner surface of elongate adjacent catheter body so that flushing fluid entering the central lumen of the elongate inner catheter body through the single injection port will flow radially outwardly through the one or more flush ports to enter the annular lumen.

29. The multi-catheter system of claim 23, wherein the adjacent catheter is an intermediate catheter or an outer catheter.

30. The multi-catheter system of claim 23, wherein the distal region of the inner flushing catheter includes a tapered distal end.

31. The multi-catheter system of claim 24, wherein the adjacent catheter has at least one flush segment and both the inner flushing catheter and the adjacent catheter are nested within a lumen of an outer catheter.

32. The multi-catheter system of claim 26, wherein an intermediate catheter is nested between the adjacent catheter and the outer catheter and the intermediate catheter includes at least one flush segment.

33. The multi-catheter system of claim 23, wherein the inner flushing catheter includes a first fastening mechanism for interlocking to a larger catheter and second fastening mechanism for interlocking to a smaller catheter, whereby interlocked smaller catheters seal the proximal end of larger catheters.

34. The multi-catheter system of claim 23, wherein inner flushing catheter includes two or more flush segments.

35. The multi-catheter system of claim 23, wherein the flush segment of the inner flushing catheter is located in the proximal region, the central region, the distal region, or a combination of two or more such regions of the catheter body.

36. The multi-catheter system of claim 26, wherein the flush segment of the adjacent catheter is located in the proximal region, the central region, the distal region, or a combination of two or more such regions of the catheter body.
